(19) United States
(12) Patent Application Publication
(10) Pub. No.: US 2016/0257664 A1
(43) Pub. Date: Sep. 8, 2016

(54) NOVEL COMPOUNDS

(71) Applicant: GLAXOSMITHKLINE LLC, Wilmington, DE (US)

(72) Inventors: Veronique BIRAULT, Stevenage, Hertfordshire (GB); Amanda Jennifer CAMPBELL, Stevenage, Hertfordshire (GB); Stephen Anthony HARRISON, Stevenage, Hertfordshire (GB); Joelle LE, Stevenage, Hertfordshire (GB)

(21) Appl. No.: 15/031,323

(22) PCT Filed: Oct. 23, 2014
(86) PCT No.: PCT/US14/61864

§ 371 Date: Apr. 22, 2016

Related U.S. Application Data
(60) Provisional application No. 61/895,470, filed on Oct. 25, 2013.

(51) Int. Cl. C07D 309/06

(52) U.S. Cl. CPC C07D 309/06; C07B 2200/13

(57) ABSTRACT

The present invention is directed to crystalline forms of a compound which has retinoid-related orphan receptor gamma (RORγ) modulator activity, processes for their preparation, pharmaceutical compositions containing the same and their use in therapy.

### FIELD OF THE INVENTION

The present invention is directed to crystalline forms of a compound which has retinoid-related orphan receptor gamma (RORγ) modulator activity. More particularly the present invention relates to crystalline forms of the compound N-(4-ethylphenyl)-3-(hydroxymethyl)-N-isobutyl-4-((tetrahydro-2H-pyran-4-yl)methoxy)benzenesulfonamide, processes for their preparation, pharmaceutical compositions containing the same and their use in therapy.

### BACKGROUND OF THE INVENTION

Retinoid-related orphan receptors (RORs) are transcription factors that form a subgroup of the nuclear receptor superfamily (Adv. Dev. Biol. 2006, 16, 313-355). This subgroup consists of three members: ROR alpha (RORα), ROR beta (RORβ) and ROR gamma (RORγ). RORα and RORβ have approximately 55% homology in the ligand binding domains to RORγ. RORs contain four principal domains shared by the majority of nuclear receptors: an N-terminal A/B domain, a DNA-binding domain, a hinge domain and a ligand binding domain.

The RORα, RORβ and RORγ genes have been mapped to human chromosomes 15q22.2, 9q21.13 and 1q21.3, respectively. Each ROR gene generates several isoforms, which differ only in their N-terminal A/B domain. To date, five splice variants have been recorded for RORγ and two isoforms of this member of the ROR family have been identified: RORγ1 and RORγ2 (also known as RORγt). RORγ is a term used to describe RORγ1 and/or RORγt.

While RORγ1 is expressed in a variety of tissues including thymus, muscle, kidney and liver, RORγt is exclusively expressed in the cells of the immune system and has a critical role in thymopoiesis, development of several secondary lymphoid tissues and Th17 lineage specification.

RORγt has been identified as a key regulator of Th17 cell differentiation (A. Jetten, Nuclear Receptor Signalling 2009, 7, 1-32). Th17 cells are a recently discovered subset of T helper cells which preferentially produce cytokines IL-17A, IL-17F, IL-21 and IL-22. RORγt also induces transcription of the gene encoding IL-17A and IL-17F in naive CD4⁺ T helper cells, iNKT and NKT (Mucosal Immunol. 2009, 2(5), 383-392; J. Immunol. 2008, 180, 5167-5171), γδT cells (Am. J. Respir. Crit. Care Med. 2010, 182, 464-476), CD8⁺ T cells (J. Leukocyte Biol. 2007, 82, 354-360), group 3 Innate Lymphoid Cells (Nature Rev. Immunol. 2013, 13, 145-149) and finally CD4⁻CD8⁻TCRα62 ⁺ T cells (J. Immunol. 2008, 181, 8761-8766). Additional immune cells such as eosinophils, neutrophils and macrophages can also be a source of IL-17A in allergic inflammation related to asthma (J. Allergy Clin. Immunol. 2001, 108, 430-438; J. Immunol. 2008, 181, 6117-6124; Immunity2004, 21, 467-476).

Th17 cells and their products have been shown to be associated with the pathology of a number of human inflammatory and autoimmune disorders. IL-17A and IL-17F are implicated in numerous immune and inflammatory responses primarily as pro-inflammatory regulators inducing the expression of cytokines, chemokines, adhesion molecules, mucin genes and growth factors. There is emerging evidence that an increase in Th17 cytokines are closely associated with a range of chronic inflammatory diseases such as rheumatoid arthritis (Curr. Opin. Investig. Drugs 2009, 10, 452-462), multiple sclerosis (Allergol. Int 2008, 57(2), 115-120), inflammatory bowel diseases (J. Inflamm. Res. 2010, 3, 33-44), glomerulonephritis (J Am Soc Nephrol. December 2009; 20(12): 2518-2524), uveitis (Nat Med. 2007 Jun;13(6):711-8), psoriasis (Sci. Trans/. Med. 2010, 2(52)), psoriatic arthritis (Clin Rev Allergy Immunol. 2013 April; 44(2):183-93), behcet's disease (Clin Exp Rheumatol. 2011 July-August;29(4 Suppl 67):571-6), Sjogren's syndrome (Ann Rheum Dis. 2014 Feb. 26.), dry eye disease (Mucosal Immunol. July 2009; 2(4): 375-376), atopic dermatitis (J. Investigative Dermatol. 2008, 128, 2625-2630), acne (PLoS ONE 2014, 9(8), e105238-e105238) and lung diseases (Prog. Respir. Res. Base 12010, 39, 141-149; Resp. Research 2010, 11 (78), 1-11).

PCT patent application PCT/EP2013/058666 discloses a series of sulphonamide derivatives as RORγ modulators. In particular the compound N-(4-ethylphenyl)-3-(hydroxymethyl)-N-isobutyl-4-((tetrahydro-2H-pyran-4-yl)methoxy)benzenesulfonamide, that is to say the compound having the formula

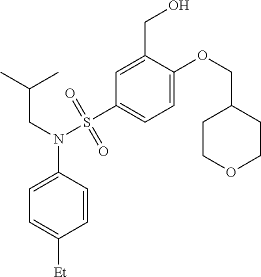 [SMILES: CCc1ccc(N(CC(C)C)S(=O)(=O)c2ccc(OCC3CCOCC3)c(CO)c2)cc1]

is disclosed therein as Example 124. The PCT publication was published on 31 Oct. 2013 as publication WO2013/160418, and is hereby incorporated by reference. The product of the preparation described in this patent application is a white foam. Therefore there exists a need for a form of N-(4-ethylphenyl)-3-(hydroxymethyl)-N-isobutyl-4-((tetrahydro-2H-pyran-4-yl)methoxy)benzenesulfonamide which is suitable for the development of a pharmaceutical product.

### SUMMARY OF THE INVENTION

In a first aspect of the present invention there is provided a crystalline form of the compound N-(4-ethylphenyl)-3-(hydroxymethyl)-N-isobutyl-4-((tetrahydro-2H-pyran-4-y1)methoxy)benzenesulfonamide.

In a second aspect of the present invention there is provided a pharmaceutical composition comprising a crystalline form of the compound N-(4-ethylphenyl)-3-(hydroxymethyl)-N-isobutyl-4-((tetrahydro-2H-pyran-4-yl)methoxy)benzenesulfonamide and one or more pharmaceutically acceptable excipients.

In a third aspect of the present invention there is provided a crystalline form of the compound N-(4-ethylphenyl)-3-(hydroxymethyl)-N-isobutyl-4-((tetrahydro-2H-pyran-4-yl)methoxy)benzenesulfonamide for use in therapy, particularly for use in the treatment of inflammatory, metabolic and autoimmune diseases mediated by RORγ.

In a fourth aspect of the present invention there is provided a method of treatment of inflammatory, metabolic and autoimmune diseases mediated by RORγ which comprises administering to a subject in need thereof a crystalline form of the compound N-(4-ethylphenyl)-3-(hydroxymethyl)-N-isobutyl-4-((tetrahydro-2H-pyran-4-yl)methoxy)benzenesulfonamide.

### DESCRIPTION OF THE DRAWINGS

FIG. 1: Showing XRPD data of a crystalline form of N-(4-ethylphenyl)-3-(hydroxymethyl)-N-isobutyl-4-((tetrahydro-2H-pyran-4-yl)methoxy)benzenesulfonamide as prepared in Example 1 (herein referred to as “anhydrous form 1”).

FIG. 2: Showing the DSC thermogram of a crystalline form of N-(4-ethylphenyl)-3-(hydroxymethyl)-N-isobutyl-4-((tetrahydro-2H-pyran-4-yl)methoxy)benzenesulfonamide as prepared in Example 1 (herein referred to as “anhydrous form 1”).

FIG. 3: Showing XRPD data of a crystalline form of N-(4-ethylphenyl)-3-(hydroxymethyl)-N-isobutyl-4-((tetrahydro-2H-pyran-4-yl)methoxy)benzenesulfonamide as prepared in Example 2 (herein referred to as “hydrate 1”).

FIG. 4: Showing the DSC thermogram of a crystalline form of N-(4-ethylphenyl)-3-(hydroxymethyl)-N-isobutyl-4-((tetrahydro-2H-pyran-4-yl)methoxy)benzenesulfonamide as prepared in Example 2 (herein referred to as “hydrate 1”).

FIG. 5: Showing XRPD data of a crystalline form of N-(4-ethylphenyl)-3-(hydroxymethyl)-N-isobutyl-4-((tetrahydro-2H-pyran-4-yl)methoxy)benzenesulfonamide as prepared in Example 3 (herein referred to as “hydrate 2”).

FIG. 6: Showing the DSC thermogram of a crystalline form of N-(4-ethylphenyl)-3-(hydroxymethyl)-N-isobutyl-4-((tetrahydro-2H-pyran-4-yl)methoxy)benzenesulfonamide as prepared in Example 3 (herein referred to as “hydrate 2”).

### DETAILED DESCRIPTION OF THE INVENTION

In a first aspect, the present invention provides a crystalline form of N-(4-ethylphenyl)-3-(hydroxymethyl)-N-isobutyl-4-((tetrahydro-2H-pyran-4-yl)methoxy)benzenesulfonamide.

It will be appreciated that the compound N-(4-ethylphenyl)-3-(hydroxymethyl)-N-isobutyl-4-((tetrahydro-2H-pyran-4-yl)methoxy)benzenesulfonamide may exist in a number of different crystalline forms. Said crystalline forms include solvates (e.g. hydrates) and anhydrate forms. Such forms can be characterized and differentiated using a number of conventional analytical techniques, including, but not limited to, X-ray powder diffraction (XRPD) patterns, infrared (IR) spectra, Raman spectra, differential scanning calorimetry (DSC), thermogravimetric analysis (TGA) and solid state nuclear magnetic resonance (SSNMR).

In one embodiment there is provided an anhydrous crystalline form of N-(4-ethylphenyl)-3-(hydroxymethyl)-N-isobutyl-4-((tetrahydro-2H-pyran-4-yl)methoxy)benzenesulfonamide.

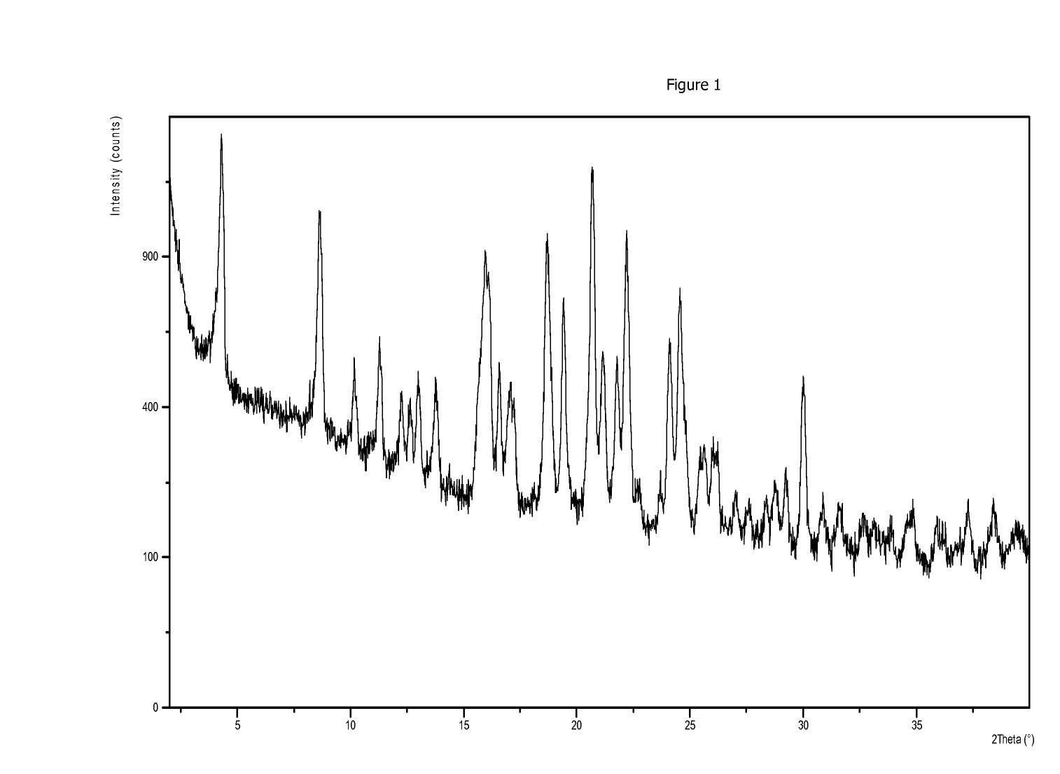

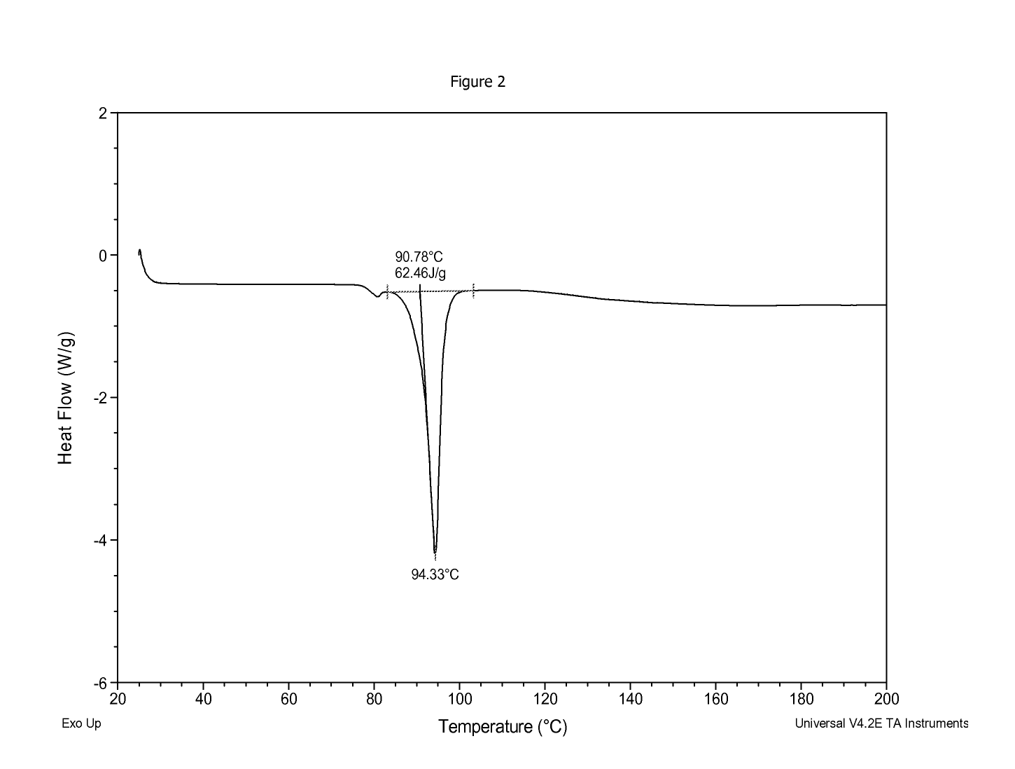

In a particular embodiment there is provided an anyhydrous crystalline form of N-(4-ethylphenyl)-3-(hydroxymethyl)-N-isobutyl-4-((tetrahydro-2H-pyran-4- yl)methoxy)benzenesulfonamide (“anhydrous form 1”) characterised by substantially the same X-ray powder diffraction (XRPD) pattern as shown in FIG. 1, wherein the XRPD pattern is expressed in terms of 2 theta angles and obtained with a diffractometer using copper Kα-radiation using procedures described herein and/or substantially the same differential scanning calorimetry (DSC) thermograms as shown in FIG. 2 wherein the DSC was performed at a scan rate of 15° per minute using procedures described herein. The XRPD of anhydrous form 1 shows 2 theta angle peaks as provided in the list in Table 1 with characteristic 2 theta angle peaks at 4.3±0.1, 8.6±0.1 and 10.2±0.1. The DSC of anhydrous form 1 shows a sharp melting endotherm with an onset temperature of approximately 90.8° C.

In a further embodiment there is provided a hydrated crystalline form of N-(4-ethylphenyl)-3-(hydroxymethyl)-N-isobutyl-4-((tetrahydro-2H-pyran-4-yl)methoxy)benzenesulfonamide.

Hydrated crystalline forms of the compound N-(4-ethylphenyl)-3-(hydroxymethyl)-N-isobutyl-4-((tetrahydro-2H-pyran-4-yl)methoxy)benzenesulfonamide include a hemi-hydrate, a hydrate (1:1 stoichiometry) and a di-hydrate.

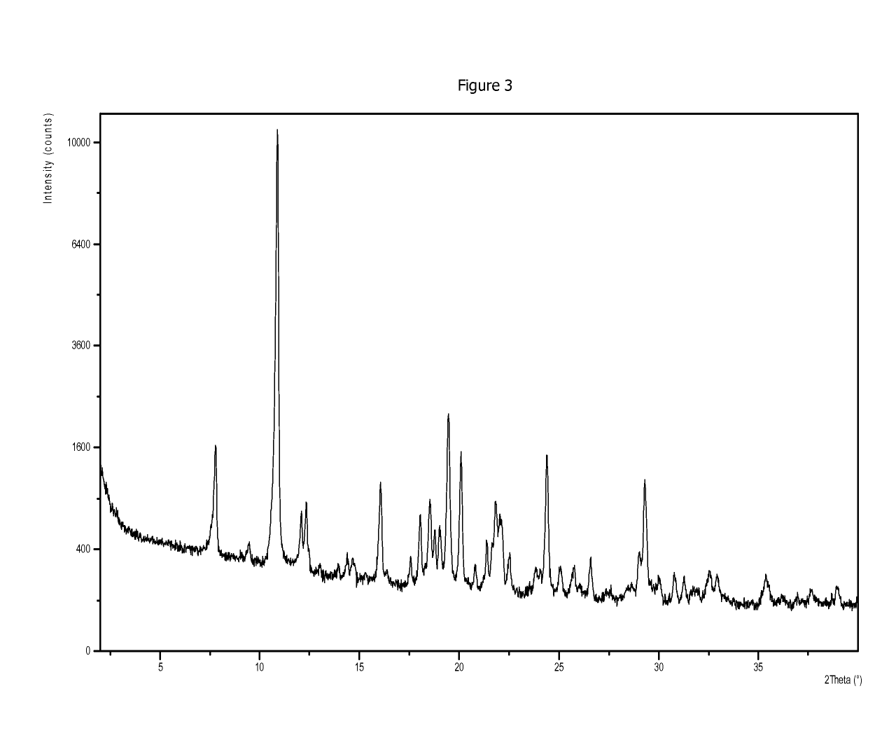

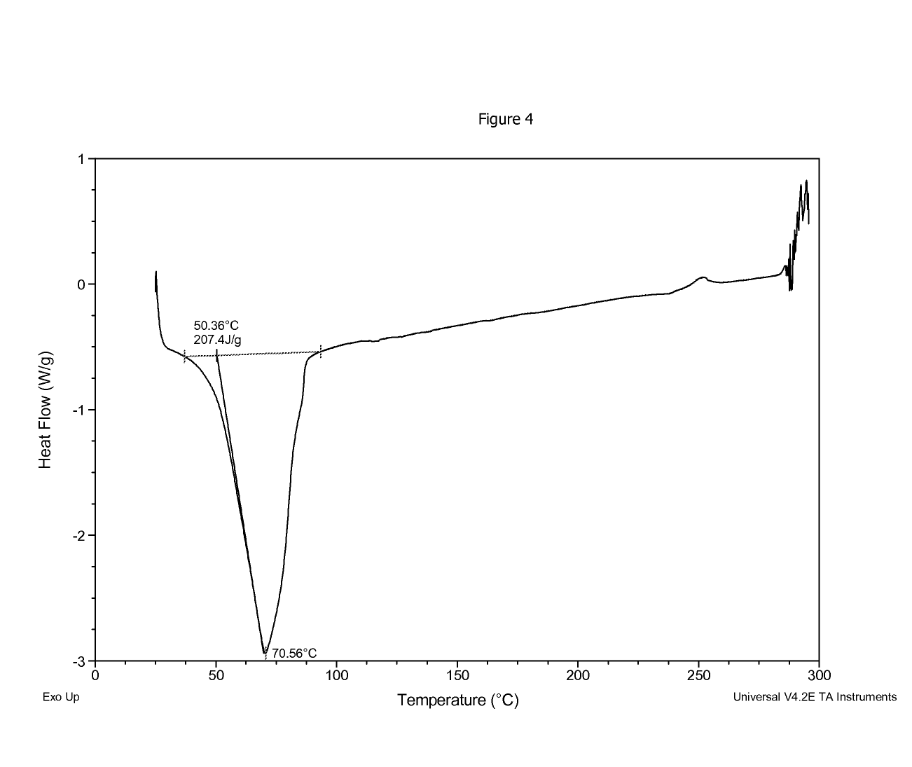

In a particular embodiment there is provided a hydrated crystalline form of N-(4-ethylphenyl)-3-(hydroxymethyl)-N-isobutyl-4-((tetrahydro-2H-pyran-4-yl)methoxy)benzenesulfonamide (“hydrate 1”) characterised by substantially the same X-ray powder diffraction (XRPD) pattern as shown in FIG. 3, wherein the XRPD pattern is expressed in terms of 2 theta angles and obtained with a diffractometer using copper Kα-radiation using procedures described herein and/or substantially the same differential scanning calorimetry (DSC) thermograms as shown in FIG. 4 wherein the DSC was performed at a scan rate of 15° per minute using procedures described herein. The XRPD of hydrate 1 shows 2 theta angle peaks as provided in the list in Table 1 with characteristic 2 theta angle peaks at 7.8±0.1 and 20.1±0.1. The DSC of hydrate 1 shows a melting endotherm with an onset temperature of approximately 50° C.

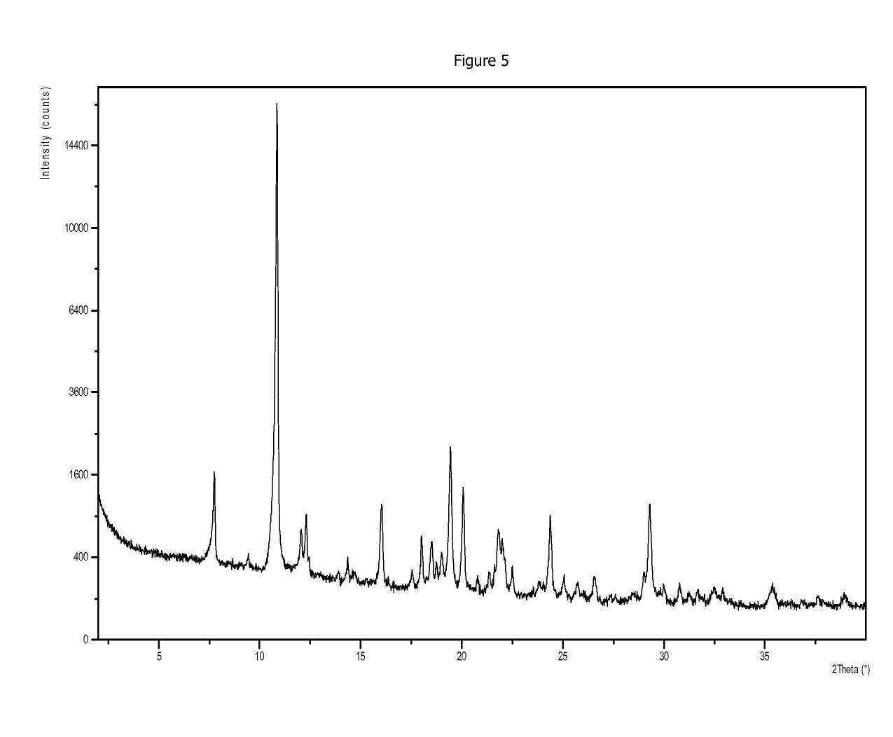

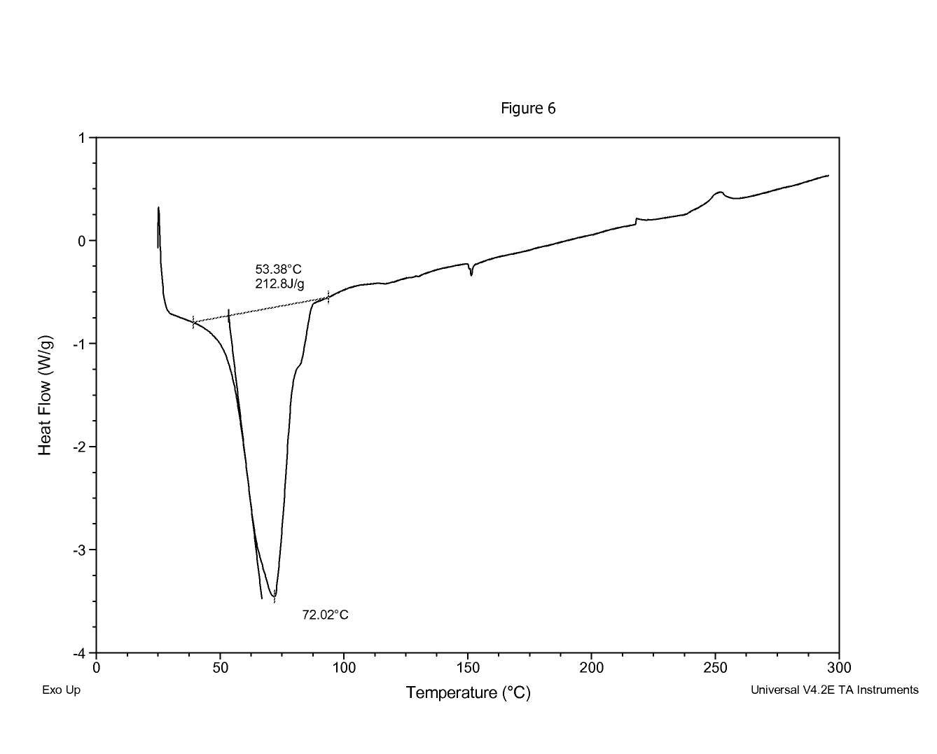

In a further embodiment, the present invention provides a crystalline form of N-(4-ethylphenyl)-3-(hydroxymethyl)-N-isobutyl-4-((tetrahydro-2H-pyran-4- yl)methoxy)benzenesulfonamide (“hydrate 2”) characterised by substantially the same X-ray powder diffraction (XRPD) pattern as shown in FIG. 5, wherein the XRPD pattern is expressed in terms of 2 theta angles and obtained with a diffractometer using copper Kα-radiation using procedures described herein and/or substantially the same differential scanning calorimetry (DSC) thermograms as shown in FIG. 6 wherein the DSC was performed at a scan rate of 15° per minute using procedures described herein. The XRPD of hydrate 2 shows characteristic 2 theta angle peaks at 7.8±0.1 and 20.1±0.1. The DSC of hydrate 2 shows a melting endotherm with an onset temperature of approximately 53.4° C.

Hydrates 1 and 2 form part of a group of structurally similar solvates (herein after referred to a “Class A solvates”). The XRPD of class A solvates shows characteristic 2 theta angle peaks at 7.8±0.2 and 20.1±0.2.

The compound N-(2,2-dimethylpropyl)-6-{3-fluoro-5-[(3-isoxazolylamino)carbonyl]-2-methylphenyl}-3-pyridinecarboxamide can be prepared by procedures described herein as represented by the methods outlined in Scheme 1.

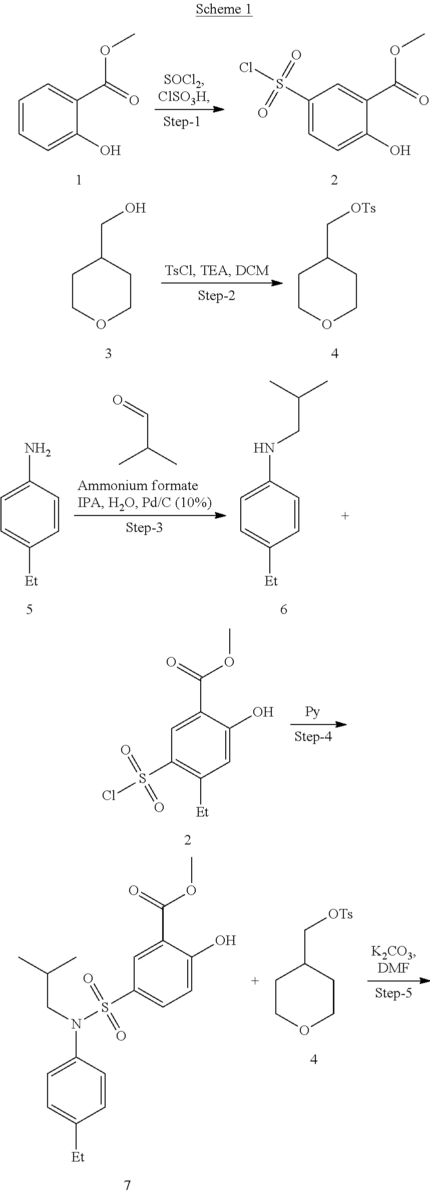

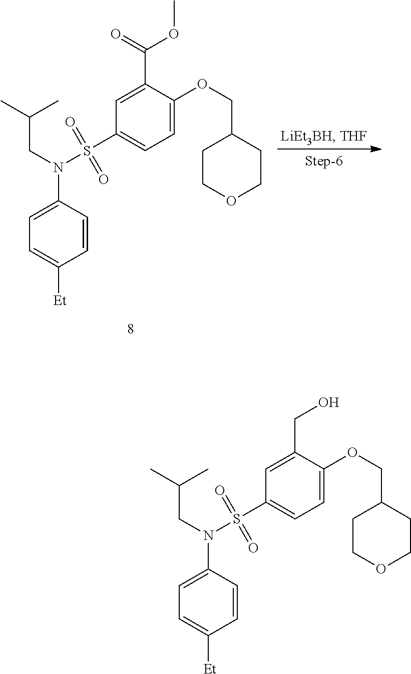 [SMILES: CCc1ccc(N(CC(C)C)S(=O)(=O)c2ccc(OCC3CCOCC3)c(C(=O)OC)c2)cc1 ; CCc1ccc(N(CC(C)C)S(=O)(=O)c2ccc(OCC3CCOCC3)c(CO)c2)cc1]

As used herein, the term “RORγ” refers to all isoforms of this member of the ROR family, including RORγ1 and RORγt.

As used herein, the term “RORγ modulator” refers to a chemical compound of formula (I) that inhibits, either directly or indirectly, the activity of RORγ. RORγ modulators include antagonists and inverse agonists of RORγ.

### Utility

The compound N-(4-ethylphenyl)-3-(hydroxymethyl)-N-isobutyl-4-((tetrahydro-2H-pyran-4-yl)methoxy)benzenesulfonamide is a modulator of RORγ and can be useful in the treatment of inflammatory, metabolic and autoimmune diseases mediated by RORγ such as asthma, chronic obstructive pulmonary disease (COPD) and bronchitis, allergic diseases, such as allergic rhinitis and atopic dermatitis, cystic fibrosis, lung allograph rejection, multiple sclerosis, rheumatoid arthritis, juvenile Rheumatoid arthritis, Osteoarthritis, ankylosing spondylitis, systemic lupus erythematosus, acne, psoriasis, Hashimoto's disease, pancreatisis, autoimmune diabetes, autoimmune ocular disease, ulcerative colitis, Crohn's disease, inflammatory bowel disease (IBS), inflammatory bowel syndrome (IBD), Sjorgen's syndrome, optic neuritis, type I diabetes, neuromyelitis optica, Myastehnia Gravis, uveitis, Guillain-Barre syndrome, psoriatic arthritis, Graves' disease and scleritis.

In a further aspect, the present invention also provides for a crystalline form of the compound N-(4-ethylphenyl)-3-(hydroxymethyl)-N-isobutyl-4-((tetrahydro-2H-pyran-4-yl)methoxy)benzenesulfonamide for use in therapy.

In a further aspect, the present invention also provides a crystalline form of the compound N-(4-ethylphenyl)-3-(hydroxymethyl)-N-isobutyl-4-((tetrahydro-2H-pyran-4-yl)methoxy)benzenesulfonamide for use in the treatment of inflammatory, metabolic and autoimmune diseases mediated by RORγ.

In one embodiment there is provided a crystalline form of the compound N-(4-ethylphenyl)-3-(hydroxymethyl)-N-isobutyl-4-((tetrahydro-2H-pyran-4-yl)methoxy)benzenesulfonamide for use in the treatment of psoriasis.

In another embodiment there is provided a crystalline form of the compound N-(4-ethylphenyl)-3-(hydroxymethyl)-N-isobutyl-4-((tetrahydro-2H-pyran-4-yl)methoxy)benzenesulfonamide for use in the treatment of atopic dermatitis.

In another embodiment there is provided a crystalline form of the compound N-(4-ethylphenyl)-3-(hydroxymethyl)-N-isobutyl-4-((tetrahydro-2H-pyran-4-yl)methoxy)benzenesulfonamide for use in the treatment of acne.

In a further aspect, the present invention is directed to a method of treatment of an inflammatory, metabolic or autoimmune disease mediated by RORγ, which comprises administering to a subject in need thereof, a safe and therapeutically effective amount of a crystalline form of the compound N-(4-ethylphenyl)-3-(hydroxymethyl)-N-isobutyl-4-((tetrahydro-2H-pyran-4-yl)methoxy)benzenesulfonamide.

In a further aspect, the present invention is directed to a method for the treatment of psoriasis, which comprises administering to a subject in need thereof, a safe and therapeutically effective amount of a crystalline form of the compound N-(4-ethylphenyl)-3-(hydroxymethyl)-N-isobutyl-4-((tetrahydro-2H-pyran-4-yl)methoxy)benzenesulfonamide.

In a further aspect, the present invention is directed to a method for the treatment of acne, which comprises administering to a subject in need thereof, a safe and therapeutically effective amount of a crystalline form of the compound N-(4-ethylphenyl)-3-(hydroxymethyl)-N-isobutyl-4-((tetrahydro-2H-pyran-4-yl)methoxy)benzenesulfonamide.

In a further aspect, the present invention is directed to a method for the treatment of atopic dermatitis, which comprises administering to a subject in need thereof, a safe and therapeutically effective amount of a crystalline form of the compound N-(4-ethylphenyl)-3-(hydroxymethyl)-N-isobutyl-4-((tetrahydro-2H-pyran-4-yl)methoxy)benzenesulfonamide.

As used herein, the term “treatment” refers to prophylaxis of the condition, ameliorating or stabilising the specified condition, reducing or eliminating the symptoms of the condition, slowing or eliminating the progression of the condition, and preventing or delaying reoccurrence of the condition in a previously afflicted patient or subject.

As used herein, the term “therapeutically effective amount” refers to the quantity of a crystalline form of the compound N-(4-ethylphenyl)-3-(hydroxymethyl)-N-isobutyl-4-((tetrahydro-2H-pyran-4-yl)methoxy)benzenesulfonamide which will elicit the desired biological response in an animal or human body.

As used herein, the term “subject” refers to an animal or human body.

### Pharmaceutical Development

A crystalline form of the compound N-(4-ethylphenyl)-3-(hydroxymethyl)-N-isobutyl-4-((tetrahydro-2H-pyran-4-yl)methoxy)benzenesulfonamide will normally, but not necessarily, be formulated into pharmaceutical compositions prior to administration to a patient. Accordingly, in another aspect the invention is directed to pharmaceutical compositions comprising a crystalline form of the compound N-(4-ethylphenyl)-3-(hydroxymethyl)-N-isobutyl-4-((tetrahydro-2H-pyran-4-yl)methoxy)benzenesulfonamide and one or more pharmaceutically-acceptable excipients.

Suitable pharmaceutical compositions may be prepared using techniques and methods known to those skilled in the art. Some of the methods commonly used in the art are described in Remington's Pharmaceutical Sciences (Mack Publishing Company).

A pharmaceutical composition of a crystalline form of the compound N-(4-ethylphenyl)-3-(hydroxymethyl)-N-isobutyl-4-((tetrahydro-2H-pyran-4-yl)methoxy)benzenesulfonamide may be formulated for administration by any appropriate route, for example by the inhaled, nasal, oral (including buccal or sublingual), topical (including buccal, sublingual, transdermal, epicutaneous) or parenteral (subcutaneous, intramuscular, intravenous, intradermal) route. Thus, a pharmaceutical composition of a crystalline form of the compound N-(4-ethylphenyl)-3-(hydroxymethyl)-N-isobutyl-4-((tetrahydro-2H-pyran-4-yl)methoxy)benzenesulfonamide may be formulated as, for example, a solution or suspension (aqueous or non-aqueous), tablet, capsule, powder, granule, lozenge, lotion, cream, ointment, gel, foam or reconstitutable powder depending on the particular route of administration. Such pharmaceutical compositions may be prepared by any method known in the art of pharmacy, for example by bringing into association the active ingredient with the excipient(s).

In one embodiment the pharmaceutical composition is adapted for oral administration.

In a further embodiment the the pharmaceutical composition is adapted to topical administration.

A crystalline form of the compound N-(4-ethylphenyl)-3-(hydroxymethyl)-N-isobutyl-4-((tetrahydro-2H-pyran-4-yl)methoxy)benzenesulfonamide can be administered in a daily dose (for an adult patient) of, for example, an oral or parenteral dose of 0.01 mg to 3000 mg per day or 0.5 to 1000 mg per day, or a nasal or inhaled dose of 0.001 to 50 mg per day or 0.01 to 5 mg per day. This amount may be given in a single dose per day or more usually in a number (such as two, three, four, five or six) of sub-doses per day such that the total daily dose is the same.

It should be understood that in addition to the ingredients particularly mentioned above, the pharmaceutical compositions may include other agents conventional in the art having regard to the type of formulation in question. A crystalline form of the compound N-(4-ethylphenyl)-3-(hydroxymethyl)-N-isobutyl-4-((tetrahydro-2H-pyran-4- yl)methoxy)benzenesulfonamide may be used in combination with one or more other therapeutic agents, selected from the group consisting of β₂-adrenoreceptor agonists, anti-inflammatory agents (e.g. corticosteroids and NSAID's) and anticholinergic agents.

The invention thus provides in a further aspect a combination comprising a crystalline form of the compound N-(4-ethylphenyl)-3-(hydroxymethyl)-N-isobutyl-4-((tetrahydro-2H-pyran-4-yl)methoxy)benzenesulfonamide and one or more other therapeutic agents.

### EXPERIMENTAL DETAILS

### Example 1

A crystalline form of N-(4-ethylphenyl)-3-(hydroxymethyl)-N-isobutyl-4-((tetrahydro-2H-pyran-4-yl)methoxy)benzenesulfonamide (anhydrous form 1)

The title compound was prepared by the following sequence of reaction steps.

### Step 1

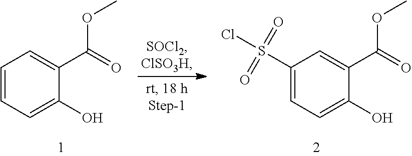

Procedure: To a stirred mixture of SOCl₂ (7.76 g) and CISO₃H (22 g), was added 10 g of compound 1 (commercially available, Sigma-Aldrich) drop-wise at 0° C. in 30 minutes. The reaction was allowed to warm to RT and then stirred for 18 hrs at RT. The reaction was monitored by TLC.

Work up: On completion of the reaction by TLC (20% methanol in DCM, product R_{F}=0.25), the reaction mixture was poured into ice cold water (200 mL) and stirred for 30 minutes at RT. The solid by filtered by bucker funnel and washed with water (200 ml), and dried under vacuum to afford 15 g of compound 2 as a white solid yield 93%.

Characterization: ¹H NMR: (400 MHz, CDCl₃): 11.54 (bs, 1H), 8.571-8.563 (dd, J=3.2 Hz, 1H), 8.104-8.075 (d, J=1.2Hz, 1H), 7.261-7.171 (m, 1H), 4.072-4.04 (1s, 3H).

### Step 2

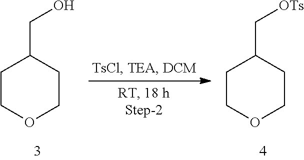 [SMILES: OCC1CCOCC1 ; Cc1ccc(S(=O)(=O)OCC2CCOCC2)cc1]

Procedure: To a stirred solution of compound 3 (commercially available, Alfa Aesar) (20 g) in dry dichloromethane (200 ml), was added p-tolunesulphonylchloride (36 gm) at RT, followed by triethylamine 26 mL) at RT under a nitrogen atmosphere. The reaction was stirred for 18 hr at RT and was monitored by TLC.

Work up: On completion of the reaction by TLC (50% ethyl acetate in hexane, product R_{F}=0.5), the reaction mixture was quenched with ice cold water, extracted with ethyl acetate (2×500 mL) washed with brine solution (2×250 mL) and dried over anhydrous Na₂SO₄. The solvent was removed under reduced pressure to afford 30 g of compound 4 as a white solid yield 65%.

Characterization: ¹HNMRδ: (400 MHz, CDCl3): 7.798-7.778, (dd, J=8 Hz, 2H), 7.364-7.344 (dd, J=8 Hz, 2H), 3.957-3.930 (d, 2H), 3.867-3.849 (d, 2H), 1.956.922 (m, 1H), 1.602-1.573 (d, 2H), 1.325-1.220 (d, 2H).

### Step 3

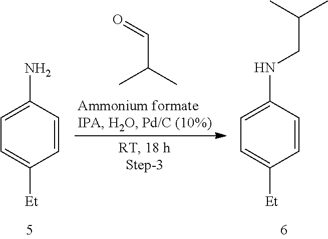

Procedure: To a stirred solution of Compound 5 (commercially available, Sigma-Aldrich) (10 g) in a mixture of isopropanol (90 mL) and water (10 mL) was added (7.73 g) isopropylbutyaldehde and (6.76 g) ammoniumformate followed by 10% Pd/C at RT. The reaction was stirred for 18 h at RT.

Work up: On completion of the reaction by TLC (10% ethyl acetate in hexane , product R_{f}=0.65), the reaction mixture was quenched with ice cold water(200 mL), extracted with ethyl acetate (2×500 mL) washed with brine solution (2×250 mL) and dried over anhydrous Na₂SO₄. The solvent was removed under reduced pressure to afford 12 g of the crude product.

Purification: The crude product was purified by column chromotography using 100-200 silcagel by eluting solvent 2% ethyl acetate in hexane to afford 10 g of compound 6 as a colourless liquid yield 71%.

Characterization: ¹HNMR (400 MHz, CDCl₃), 3.57 (bs, 1H), 7.01-6.99 (dd, J=7.2 Hz, 2H), 6.563-6.536 (m, J=6.8 Hz, 2H), 2.291-2.898 (dd, 2H), 2.56-2.503 (t, 2H), 1.92-1823 (p, 1H), 1.202-1.164 (t, 3H), 0.979-0.962 (d, 6H).

### Step 4

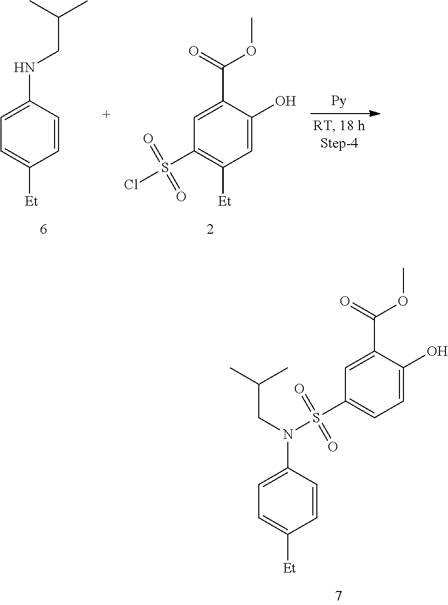 [SMILES: CCc1ccc(NCC(C)C)cc1 ; CCc1cc(O)c(C(=O)OC)cc1S(=O)(=O)Cl ; CCc1ccc(N(CC(C)C)S(=O)(=O)c2ccc(O)c(C(=O)OC)c2)cc1]

Procedure: To a stirred solution of Compound 6 (10 g) in dry pyridine (100 mL) was added Compound 2 (15.53 g) at RT under nitrogen atmosphere, and the stirring continued at RT for 18 hrs.

Work up: On completion of the reaction by TLC (10% ethyl acetate in hexane, product R_{F}=0.35), reaction mixture poured into ice-cold water (200 mL), stirred for 2 h at RT, the solid was filtered by bucker funnel, washed with water (100 mL) and dried, under vacuum to afford compound 7 as a pink colour solid yield 84%.

Characterization: ¹HNMRδ (400 MHz, CDCl3), 11.207 (bs,1H), 8.106-8.100, (m, 1H), 7.558-7.552 (m, 1H), 7.141-7.120 (m, 2H), 7.016-7.01 (m, 1H), 6.970-6.949 (m, 2H), 3.96-3.95, (s, 3H), 3.294-3.276, (d, 2H), 2.669-2.2612, (q, 2H), 1.615-1.564, (m, 1H), 1.23-1.22, (m, 3H), 0.962-0.90, (m, 6H).

### Step 5

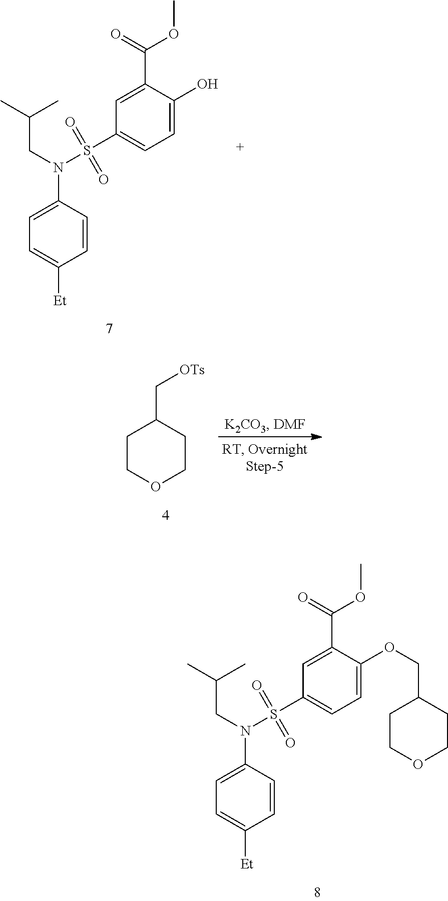 [SMILES: CCc1ccc(N(CC(C)C)S(=O)(=O)c2ccc(O)c(C(=O)OC)c2)cc1 ; Cc1ccc(S(=O)(=O)OCC2CCOCC2)cc1 ; CCc1ccc(N(CC(C)C)S(=O)(=O)c2ccc(OCC3CCOCC3)c(C(=O)OC)c2)cc1]

Procedure: To a stirred solution of Compound 7 (12 g) and Compound 4 in dry DMF (120 mL) was added K₂CO₃ (7.5 g) at RT under nitrogen atmosphere, stirred for 18 h at RT. The reaction was monitored by TLC.

Work up: On completion of the reaction by TLC (50% ethyl acetate in hexane , product R_{F}=0.65), the reaction mixture poured into ice cold water (200 mL) , extracted with ethyl acetate (2×500 mL), washed with brine solution (2×250 mL) and dried over anhydrous Na₂SO₄. The solvent was removed under reduced pressure to get to afford 15 g crude. Purification: The crude compound was purified by column chromotography using 100-200 silcagel by eluting solvent 25% ethyl acetate in hexane to afford 12 g of compound 8 as an off white solid yield 80%.

Characterization: ¹HNMRδ: (400 MHz, CDCl3): 8.034-8.028 (m, 1H), 7.594-7.566, (m, 1H), 7.137-7.115, (m, 2H), 6.996-6.933, (m, 3H), 4.058-4.030, (d, 2H), 4.02-3.97, (d, 2H), 3.92-3.86 (s, 3H), 3.85-3.471, (t, 3H), 3.288-3.270, (m, 2H), 2.66-2.458, (q, 2H), 2.192-2.125 (m, 1H), 1.83-1.79, (d, 2H), 1.572-1.525, (m, 2H), 1.25-1.211, (t,3H), 0.97-0.89, (m, 6H).

### Step 6

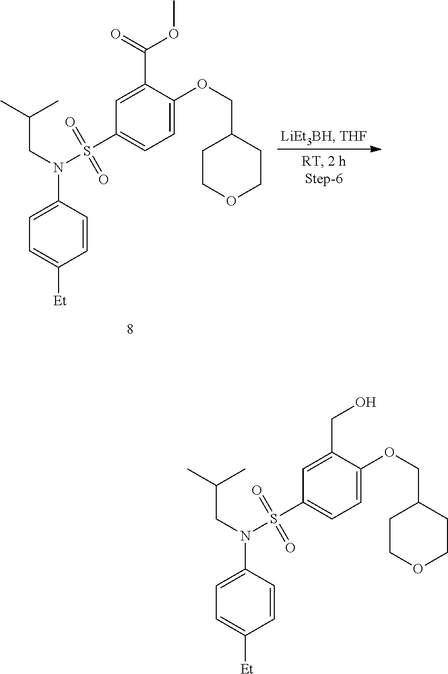 [SMILES: CCc1ccc(N(CC(C)C)S(=O)(=O)c2ccc(OCC3CCOCC3)c(C(=O)OC)c2)cc1 ; CCc1ccc(N(CC(C)C)S(=O)(=O)c2ccc(OCC3CCOCC3)c(CO)c2)cc1]

Procedure: To a stirred solution of Compound 8 (19 g) in dry THF (200 mL) was added LiEt₃BH (1M in THF, 135 mL) at 0° C., drop-wise, under nitrogen atmosphere, and stirred for 2 hr at RT. The reaction was monitored by TLC.

Work up: On completion of the reaction by TLC (30% ethyl acetate in hexane, product R_{F}=0.35), the reaction mixture was quenched with 2M HCl and NH₄ solution (1:1) 50 ml at 0° C. and the compound extracted with ethyl acetate (3×500 mL), washed with brine solution (2×500 mL) and dried over anhydrous Na₂SO₄. The solvent was removed under reduced pressure to afford 15 g of the crude product.

Purification: The crude product was purified by column chromotography using 100-200 silcagel by eluting solvent 15% ethyl acetate in hexane to afford 10.87 g of N-(4-ethylphenyl)-3-(hydroxymethyl)-N-isobutyl-4-((tetrahydro-2H-pyran-4- yl)methoxy)benzenesulfonamide (anhydrous form 1) as an off white solid yield (57.2%)

Characterization: ¹HNMRδ: (400 MHz, CDCl3), 7.579-7573, (m, 1H), 7.4567.742, (m, 1H), 7.1327112, (m, 2H), 6.978-6.957 (m, 2H), 6.858-6.836 (m, 2H), 4.68 (m, 2H), 4.057-4.049 (d, 2H), 3.92-3.904, (d, 2H), 3.494-3.431, (t, 2H), 3.281-3.262, (d, 2H), 2.665-2.606, (q, 2H), 2.144-2.061, (m, 2H), 1.77-1.733, (d, 2H), 1.598-1.480, (m, 3H,), 1.249-1.211, (t, 3H), 0.912-0.89 (s, 6H).

Preparation 1: Further preparation of the compound N-(4-ethylphenyl)-3-(hydroxymethyl)-N-isobutyl-4-((tetrahydro-2H-pyran-4-yl)methoxy)benzenesulfonamide

References to the synthetic steps and compound numbers are consistent with those of Example 1.

### LCMS Conditions:

| | |
|---|---|
| Column | BEHC18 (2.1 × 50 mm) 1.7μ |
| Mobile phase | A: 0.1% FA IN ACN |
| | B: 0.1% FA IN H₂O |
| Time % | 0/10, 0.6/10, 2/90, 5/90, 5.01/10 |
| Flow | 0.4 mL/min |
| Diluents | Acetonitrile/Water |

### Step 1

Procedure: To a stirred solution of SOCl₂ (93 mL, 1.282 mol, 1.0 eq.) and CISO₃H (245 mL, 3.681 mol, 2.87 eq.), was added Compound 1 (195 g, 1.282 mol, 1 eq.) drop wise at 0-5 ° C. (internal temp) for 1 h. The reaction mixture was allowed to warm to RT and stirred for 24 h at that temperature. The progress of the reaction was monitored by TLC.

Work up: After completion of the reaction, the reaction mixture was poured into ice cold water and stirred for 30 minutes to form a precipitated solid. The resultant this solid was filtered and dried under vacuum to afford compound 2 as a white solid (260 g, 80.9% yield).

### Step 2

Procedure: To a stirred solution of Compound 3 (145 g, 1.25 mol, 1 eq.) and triethyl amine (242.78 mL, 1.875 mol, 1.5 eq.) in dry DCM (1.5 L), was added TsCI (261.25 g, 1.375 mol, 1.1 eq.) portion wise at RT under nitrogen atmosphere for 45 minutes. The the resulting reaction mixture was then stirred at RT for 18 h. The progress of the reaction was monitored by TLC.

Work up: After completion of the reaction, the reaction mixture was diluted with ice cold water and organic layer was separated. The aqueous layer was extracted with DCM (2×1.5 L). The combined organic layer was washed with water and brine, dried over anhydrous Na₂SO₄ and concentrated under reduced pressure to get crude product as brown oil. The crude product was triturated with n-pentane to afford Compound 4 as a white solid (250.5 g, 74.07% yield). Product was confirmed by ¹H NMR and LCMS. LCMS Purity: 98.91%, 170.9 (M−H).

### Step 3

Procedure: To a stirred solution of Compound 5 (100 g, 0.826 mol, 1 eq.) in (9:1 ratio) IPA (900 mL) and water (100 mL), was added isobutyraldehyde (113 g, 1.570 mol, 1.9 eq.) and ammoniumformate (78 g, 1.239 mol, 1.5 eq.) followed by 10% Pd/C (10 g) at RT under nitrogen atmosphere. Then reaction mixture was stirred at RT for 3 hours. The progress of the reaction was monitored by TLC.

Work up: After completion of the reaction, the reaction mixture was filtered through celite bed and washed with IPA; the filtrate was concentrated under reduced pressure to obtain the crude compound. The residue was diluted with water and extracted with ethyl acetate (2×1 L). The combined organic layer was washed with brine, dried over anhydrous Na₂SO₄ and concentrated under reduced pressure to afford compound 6 as brown liquid (100.8 g, 68.8% yield). The product was confirmed by ¹H NMR and LCMS. LCMS Purity: 98.2%, 178.1 (M+H).

### Step 4

Procedure: To a stirred solution of Compound 6 (100 g, 0.5640 mol, 1 eq.) in pyridine (1 L), was added compound 2 (155.3 g, 0.6204mol, 1.1 eq.) portion wise at RT for 30 minutes. The observed internal temperature raised up to 42° C. Then the reaction mixture was stirred at RT for 18 h. The progress of the reaction was monitored by TLC.

Work up: After completion of the reaction, the reaction mixture was poured into ice cold water and stirred for 2 hours to form precipitated solid, this solid was filtered and dried under vacuum to afford Compound 7 as a brown solid (175 g, 79.2% yield). The product was confirmed by ¹H NMR and LCMS. LCMS Purity: 84.67%, 263.1 (M+H).

### Step 5

Procedure: To a stirred solution of Compound 7 (250 g, 0.6386 mol, 1 eq.) and K₂CO₃ (149.8 g, 1.085 mol. 1.7 eq.) in DMF (2.5 L), was added compound 4 (189.6 g, 0.7024 mol, 1.1 eq.) in one portion at RT. The reaction mixture was slowly heated to 100° C. and maintained for 18 h at this temperature. The progress of the reaction was monitored by TLC.

Workup: After completion of the reaction, the reaction mixture was allowed to RT, diluted with ice cold water and stirred for 20 minutes. The resultant product was extracted with ethyl acetate (2×2.5 L). The combined organic layer was washed water and brine, dried over Na₂SO₄ and evaporated under reduced pressure to obtain crude Compound 8 as a brown solid. This crude product was triturated with MTBE (500 mL) to obtain a solid which was filtered and dried under vacuum to afford pure product as off-white solid (175 g). The filtrate was concentrated under reduced pressure and purified by silica gel (60-120 mesh) column chromatography by elution with 15% ethyl acetate in petroleum ether to provide a pure product as off-white solid (49.2 g). (Total wt-224.2 g, 71.7% yield). The product was confirmed by ¹H NMR and LCMS. LCMS Purity: 98.61%, 489.9 (M+H).

### Step 6

Procedure: To a stirred solution of Compound 8 (100 g, 0.2042 mol, 1 eq.) in THF (1 L), was added super hydride (1M in THF, 715 mL, 0.715 mol, 3.5 eq.) drop wise at 0° C. for 1 h. Then reaction mixture stirred at RT for 2 h. The progress of the reaction was monitored by TLC.

Workup: After completion of the reaction, the reaction mixture was poured into (1:9 ratio) ice cold 1M HCl (200 mL), water (2 L) and stirred for 10 minutes. Saturated ammonium chloride solution (500 mL) was then added followed immediately by and ethyl acetate to the aqueous layer. The organic layer was separated, dried over Na₂SO₄ and evaporated under reduced pressure to give crude N-(4-ethylphenyl)-3-(hydroxymethyl)-N-isobutyl-4-((tetrahydro-2H-pyran-4-yl)methoxy)benzenesulfonamide as yellow oil. A total of 7 reactions (20 g, 50 g and 5×100 g) were carried out using above procedure to synthesize (501.6 g, LCMS-81%) of desired crude product. The crude product was triturated with pentane (2 L) at 0° C. for 30 minutes but no solid precipitated out. 5% ethyl acetate in petroluem ether (2 L) at 0° C. temperature was added and stirred for 10 minutes and then further stirred at RT for 1 hour. The desired solid product was filtered and dried under vacuum to provide 190 g of the desired compound as white solid. The filtrate was concentrated under reduced pressure (302 g) and using column purification, eluted with 40-50% ethyl acetate in petroleum ether, which isolated 150 g desired product as a colorless thick oil. 5% ethyl acetate in petroleum ether (500 ml) was added to the product and stirred for 1 hour. The solid product was filtered and dried under vacuum to obtain 42.3 g of the desired compound. The filtrate was concentrated and dissolved in methanol (400 mL). This was diluted with water (2 L) and stirred at RT for 2 hours. The solid product was filtered and dried under vacuum to obtain a further 80 g of white solid compound. All three solid parts (190 g, 42.3 and 80 g) were mixed in pentane (2 L) and stirred at RT for 30 minutes. The desired product was filtered and dried under vacuum at RT for 2 hours to obtain 312.3 g of the desired compound. The product was confirmed by ¹H NMR and LCMS. LCMS Purity: 98.51%, 462.27 (M+H).

### Example 2

### A Crystalline Form of N-(4-ethylphenyl)-3-(hydroxymethyl)-N-isobutyl-4-((tetrahydro-2H-pyran-4-yl)methoxy)benzenesulfonamide (“hydrate 1”)

750 μL of solvent (acetone/water in the ratio 1:4) and 40 mg of the product of Preparation 1 was combined in a 2 mL HPLC vial. The resultant slurry was then stirred for two days with cycling of temperature between 5° and 40° C. The slurry was filtered by vacuum filtration and the resultant product was analysed by XRPD and DSC.

### Example 3

### A Crystalline Form of N-(4-ethylphenyl)-3-(hydroxymethyl)-N-isobutyl-4-((tetrahydro-2H-pyran-4-yl)methoxy)benzenesulfonamide (“hydrate 2”)

The product of preparation 1 was added to 750 ηL of solvent (MEK/i-propyl ether in the ratio 1:1) in a 2 mL HPLC vial under ambient conditions until a slurry was produced. The resultant slurry was then stirred for two days with cycling of temperature between 5° and 40° C. The slurry was filtered by vacuum filtration and the resultant product was analysed by XRPD and DSC. The solution from filtration was stored at approximately 4° C. for 20 hours and any further crystalline solids produced were isolated and analysed by XRPD and DSC.

### X-Ray Powder Diffraction (XRPD)

XRPD data were acquired using either a Bruker D8 Discovery diffractometer with a HI-STAR GADDS detector or PANalytical X'Pert Pro diffractometer on Si zero-background wafers. All diffractograms were collected using a Cu Ka (45 kV/40 mA) radiation and a step size of 0.02° 2θ unless noted otherwise. Table 1 shows XRPD peak positions for two crystalline forms of the compound N-(4-ethylphenyl)-3-(hydroxymethyl)-N-isobutyl-4-((tetrahydro-2H-pyran-4-yl)methoxy)benzenesulfonamide. The experimental error in the peak positions is approximately ±0.10° 2θ. Relative peak intensities will vary due to preferred orientation. Peaks highlighted are characteristic to each form.

TABLE 1

| | |
|---|---|
| Anhydrous Form 1 | Hydrate 1 |
| (As prepared in Example 1) | (As prepared in Example 2) |

| Pos. [°2Th.] | d-spacing [Å] | Pos. [°2Th.] | d-spacing [Å] |
|---|---|---|---|
| 4.3 | 20.6 | 7.8 | 11.4 |
| 8.6 | 10.2 | 10.9 | 8.1 |
| 10.2 | 8.7 | 12.1 | 7.3 |
| 11.3 | 7.8 | 12.3 | 7.2 |
| 12.3 | 7.2 | 16.0 | 5.5 |
| 12.6 | 7.0 | 18.0 | 4.9 |
| 13.0 | 6.8 | 18.5 | 4.8 |
| 13.8 | 6.4 | 19.4 | 4.6 |
| 18.7 | 4.8 | 20.1 | 4.4 |
| 19.4 | 4.6 | 24.4 | 3.7 |
| 20.7 | 4.3 | 29.3 | 3.1 |
| 21.2 | 4.2 | | |
| 21.8 | 4.1 | | |
| 22.2 | 4.0 | | |
| 24.1 | 3.7 | | |
| 24.6 | 3.6 | | |
| 30.0 | 3.0 | | |

Hydrate 2 (as prepared in Example 3) shares some of the 2 theta angle peaks as identified for Hydrate 1. In particular, the characteristic 2 theta angle peaks at 7.8 ± 0.2 and 20.1 ± 0.2.

### Differential Scanning Calorimetry (DSC)

DSC was conducted with a TA Instruments Q100 differential scanning calorimeter equipped with an autosampler and a refrigerated cooling system under 40 mL/min N₂ purge. DSC thermograms were obtained at 15° C./min in crimped Al pans. Where used, Modulated DSC analyses were obtained by equilibrating to 0° C. and heating at 2.0° C./min with ±0.32° C. modulation every 60 seconds in crimped Al pans. Table 2 shows DSC data for three forms of the compound N-(4-ethylphenyl)-3-(hydroxymethyl)-N-isobutyl-4-((tetrahydro-2H-pyran-4-yl)methoxy)benzenesulfonamide.

TABLE 2

| Form | Onset (C. °) | Peak (C. °) | Enthalpy (J/g) |
|---|---|---|---|
| Anhydrous Form 1 | 90.8 | 94.3 | 62.5 |
| (As prepared in Example 1) | | | |
| Hydrate 1 | 50.4 | 70.6 | 207.4 |
| (As prepared in Example 2) | | | |
| Hydrate 2 | 53.4 | 72.0 | 212.8 |
| (As prepared in Example 3) | | | |

### ABBREVIATIONS

DCM dichloromethane

IPA isopropyl alcohol

MEK methyl ethyl ketone

MTBE methyl tert-butyl ether

RT room temperature

THF tetrahydrofuran

TLC thin layer chromotography

1. A crystalline form of N-(4-ethylphenyl)-3-(hydroxymethyl)-N-isobutyl-4-((tetrahydro-2H-pyran-4-yl)methoxy)benzenesulfonamide.

2. The crystalline form according to claim 1 which is an anhydrous crystalline form of N-(4-ethylphenyl)-3-(hydroxymethyl)-N-isobutyl-4-((tetrahydro-2H-pyran-4-yl)methoxy)benzenesulfonamide.

3. The crystalline form according to claim 2 having an XRPD with characteristic 2 theta angle peaks at 4.3±0.1, 8.6±0.1 and 10.2±0.1.

4. The crystalline form according to claim 2 characterised by a DSC trace with an onset temperature of approximately 90.8° C.

5. The crystalline form according to claim 1 which is a hydrated crystalline form of N-(4-ethylphenyl)-3-(hydroxymethyl)-N-isobutyl-4-((tetrahydro-2H-pyran-4-yl)methoxy)benzenesulfonamide.

6. The crystalline form according to claim 5 having an XRPD with characteristic 2 theta angle peaks at 7.8±0.1 and 20.1±0.1.

7. The crystalline form according to claim 5 characterised by a DSC trace with an onset temperature of approximately 50.4° C.

8-10. (canceled)

11. A pharmaceutical composition comprising the crystalline form of N-(4-ethylphenyl)-3-(hydroxymethyl)-N-isobutyl-4-((tetrahydro-2H-pyran-4-yl)methoxy)benzenesulfonamide according to claim 1, and a pharmaceutically acceptable excipient.

12. (canceled)

13. A method of treatment of an inflammatory, metabolic or autoimmune disease mediated by RORγ in a human in need thereof comprising administering to said human an effective amount of the crystalline form of N-(4-ethylphenyl)-3-(hydroxymethyl)-N-isobutyl-4-((tetrahydro-2H-pyran-4-yl)methoxy)benzenesulfonamide according to claim 1.

14. The method according to claim 13, wherein the inflammatory, metabolic or autoimmune disease disease mediated by RORγ is selected from asthma, chronic obstructive pulmonary disease, bronchitis, allergic diseases, allergic rhinitis, atopic dermatitis, cystic fibrosis, lung allograph rejection, multiple sclerosis, rheumatoid arthritis, juvenile Rheumatoid arthritis, Osteoarthritis, ankylosing spondylitis, systemic lupus erythematosus, acne, psoriasis, Hashimoto's disease, pancreatisis, autoimmune diabetes, autoimmune ocular disease, ulcerative colitis, Crohn's disease, inflammatory bowel disease, inflammatory bowel syndrome, Sjorgen's syndrome, optic neuritis, type I diabetes, neuromyelitis optica, Myasthenia Gravis, uveitis, Guillain-Barre syndrome, psoriatic arthritis, Graves' disease or scleritis.

15. (canceled)

16. A process for preparing the pharmaceutical composition according to claim 11, which process comprises bringing the crystalline form into association with the pharmaceutically acceptable excipient.

17. A process for preparing the crystalline form of N-(4-ethylphenyl)-3-(hydroxymethyl)-N-isobutyl-4-((tetrahydro-2H-pyran-4-yl)methoxy)benzenesulfonamide according to claim 1 comprising the steps of
(a) adding N-(4-ethylphenyl)-3-(hydroxymethyl)-N-isobutyl-4-((tetrahydro-2H-pyran-4-yl)methoxy)benzenesulfonamide to a mixture of water and an organic solvent,
(b) stirring the resulting mixture for several days while cycling the temperature, and
(c) collecting the resulting solids by filtration.

18. A method of treating psoriasis in a human in need thereof comprising administering to said human an effective amount of the crystalline form of N-(4-ethylphenyl)-3-(hydroxymethyl)-N-isobutyl-4-((tetrahydro-2H-pyran-4-yl)methoxy)benzenesulfonamide according to claim 1.

19. A method of treating acne in a human in need thereof comprising administering to said human an effective amount of the crystalline form of N-(4-ethylphenyl)-3-(hydroxymethyl)-N-isobutyl-4-((tetrahydro-2H-pyran-4-yl)methoxy)benzenesulfonamide according to claim 1.
